(19) United States
(12) Patent Application Publication
(10) Pub. No.: US 2010/0143919 A1
(43) Pub. Date: Jun. 10, 2010

(54) In vitro assay for early diagnosis of diabetes mellitus type 1

(76) Inventors: Klaus Abraham-Fuchs, Erlangen (DE); Karsten Hiltawsky, Schwerte (DE); Michael Maschke, Lonnerstadt (DE); Sebastian Schmidt, Weisendorf (DE)

Correspondence Address: HARNESS, DICKEY & PIERCE, P.L.C., P.O.BOX 8910, RESTON, VA, 20195, US

(21) Appl. No.: 12/591,915

(22) Filed: Dec. 4, 2009

(30) Foreign Application Priority Data
Dec. 8, 2008 (DE) 10 2008 061 013.5

(51) Int. Cl. C12Q 1/68; G01N 33/68

(52) U.S. Cl. 435/6; 436/86

(57) ABSTRACT

An in vitro assay is disclosed for early diagnosis of insulin-dependent diabetes mellitus type 1. In at least one embodiment, the assay includes i) determining susceptibility markers for diabetes mellitus type 1 in a biological sample from a symptom-free individual; ii) determining inflammatory markers in the same or in a further biological sample from the individual; iii) determining the presence or the risk of developing insulin-dependent diabetes mellitus type 1 on the basis of the values obtained in i) and ii); and, possibly, iv) performing step ii) after a defined time interval or v) performing an imaging method for the pancreas. At least one embodiment is further directed to the use of this assay for monitoring the progression of a diabetes mellitus type 1 disease.

### PRIORITY STATEMENT

The present application hereby claims priority under 35 U.S.C. §119 on German patent application number DE 10 2008 061 013.5 filed Dec. 8, 2008, the entire contents of which are hereby incorporated herein by reference.

### FIELD

At least one embodiment of the invention generally relates to an in vitro assay or an in vitro assay method or an in vitro method for early diagnosis of insulin-dependent diabetes mellitus type 1. In particular, at least one embodiment of the invention relates to an in vitro assay in order to detect in a timely fashion the occurrence of diabetes mellitus type 1 in high risk patients such as children. At least one embodiment of the invention furthermore generally relates to the use of this assay for monitoring the progression of a diabetes mellitus type 1 disease.

### BACKGROUND

Diabetes mellitus is a very common disease which is of great socio-economic significance. Diabetes mellitus is the most common endocrine disorder in humans and is characterized by a relative or absolute insulin deficiency. A distinction is made between type 1 diabetes and type 2 diabetes. Diabetes mellitus type 1 is insulin-dependent diabetes, characterized by a gradual depletion of the insulin secretion produced naturally in the body up to an absolute lack of insulin.

Currently, there is no cure for this type of diabetes. The only treatment option is life-long insulin substitution. In addition, there is also type 2 diabetes which usually occurs at an advanced age and is characterized by insulin secretion obtained in a limited fashion or even by increased insulin secretion, an insulin sensitivity of the tissue which has a delayed onset and is reduced, and, possibly, by inclusion of contrainsular factors, and therefore this thereby results in a relative lack of insulin. This type of diabetes can often be controlled by weight loss, dieting and possibly oral antidiabetic agents.

In Germany, the risk of developing type 1 diabetes mellitus is approximately 0.012% to 0.4%. If a family member has already developed type 1 diabetes, the risk is approximately 10 times as high.

The pathophysiological basis of diabetes mellitus type 1 is a destruction of the insulin-producing islet cells in the pancreas, which often occurs in patients in early childhood. It is known that this destruction is based on an autoimmune reaction against the islet cells (Langerhans cells), with there being an inflammation of the pancreas. A treatment of this autoimmune reaction at a very early stage could prevent the complete destruction of the islet cells and thus prevent the complete picture of insulin-dependent diabetes mellitus type 1.

However, all therapeutic preparations in respect thereof are only initiated at a very late stage because diabetes mellitus type 1 is usually only discovered at a late stage as a result of the symptoms caused by a high blood glucose level such as excessive urine production, increased thirst, visual disorders, unexplained weight loss, lethargy and changes in the energy metabolism. These symptoms can already be traced back to a significantly reduced insulin production. At this stage, the islet cells are usually almost completely destroyed and the process is irreversible. This therapeutic situation also impedes clinical research in this field.

These days, type 1 diabetes patients are generally only diagnosed once they are clinically conspicuous. This often occurs when the blood sugar rises so high that this results in a hyperglycemic coma.

Once diabetes has already manifested itself, it can be diagnosed easily and cost-effectively by the determination of the blood sugar. A fasting blood sugar level above 7 mmol/l is considered to be diagnostic.

However, at this point in time, the destruction of the Langerhans cells in the pancreas has already been completed, and the diabetes can no longer be treated causally. All that remains possible is a symptomatic therapy by means of a life-long supply of insulin. It is for this reason that diagnosis via the blood sugar is not suitable for initiating a causal therapy.

It is known that the acute inflammation can be detected with very high accuracy at the time of the inflammation (a few weeks) by imaging methods such as an MRI scan. However, these methods would have to be applied frequently (every couple of weeks) to a very large population in order to image the acute inflammation. It is for this reason that these methods nowadays play no role in clinical practice, but are only known to a small specialist audience because they are used in the animal model for research purposes (c.f. for example Denis M. et al., PNAS 101:34, pp. 12634-12639, 2004). Unlike in clinical practice in the case of humans, the diabetes is caused artificially in animal trials and so the time when the islet cells are inflamed is precisely known and hence the imaging examination is always performed at the right time without further measures being required. Furthermore, the animal models are normalized to the extent that all, or at least most, of the animals really do develop diabetes and so the problem of useless scans does not occur and, rather, the power of the imaging method as such can be tested.

Since the incidence of type 1 diabetes in Germany is approximately 12 per 100 000 children per year, 60 million precautionary examinations per year would have to be performed in Germany in order to diagnose 1200 new cases of diabetes, assuming, in the case of 10 million children and adolescents, an imaging examination being performed every eight weeks for early recognition. This cannot be justified economically due to the costs of the examination (˜1000 euros). Moreover, a very large number of healthy children would be subject to the risks of an examination with a contrast agent, with only a small number actually benefiting therefrom. By way of an example, a typical side effect rate in the case of i.v. injections of iron oxides is approximately 5%, with a large margin of error depending on the evaluation of the events and the formulation of the iron oxides. Severe side effects still affect a few thousandths of all examinees. In the case of an assumed severe side effect frequency of 1 in 1000 examinations, 60 000 cases of severe side effects would occur in the abovementioned example, whereas only 1200 examinees would benefit from the examination because it is only in them that diabetes is actually detected at an earlier stage.

It is for this reason that imaging methods have not yet found their way into standard clinical diagnosis of type 1 diabetes.

Furthermore, it is known that risk markers for type 1 diabetes exist. For example, carriers of the HLA DR3 or DR4 genotype have a significantly increased risk of developing diabetes. However, this risk is over the entire lifetime and does not allow statements in respect of the time of the disease becoming acute to be made; that is to say it cannot be used as the basis of a therapy decision.

Inflammatory markers for the acute inflammation are also known, for example antibodies against glutamate decarboxylase (anti-GAD). There is an increase in the concentration of these antibodies in diabetics for some time before and after the acute inflammation and this can be used as an indication for an acute inflammation. However, since the values of these markers are also regularly increased in healthy individuals (in approximately 3-4% of the healthy population), an increase in the concentration of such markers can likewise not be used as reliable criterion for initiating therapy. Thus, for example, in 100 000 randomly selected healthy children and adolescents, (0.012%) would develop diabetes in the next year, but approximately 2000-3000 (2-3%) would have an increased anti-GAD value, that is to say would incorrectly be subjected to therapy.

### SUMMARY

Therefore, the inventors have recognized that there is a need for effective methods for a timely diagnosis of an inflammation of the islet cells in the pancreas and hence for the early diagnosis of type 1 diabetes. In particular, there is a need for diagnosing such an inflammation as early as possible, that is to say as long as the insulin production is still normal or almost normal and when clinical symptoms have not yet occurred.

Thus, at least one embodiment of the present invention provides a diagnostic method which affords the possibility of diagnosing diabetes mellitus type 1 as early as possible, that is to say preferably before the occurrence of clinically manifest symptoms. The method should be able to as far as possible detect the initial stages of an inflammation of the islet cells in the pancreas. The method should be as sensitive as possible and, where possible, be able to detect early symptoms of an inflammation of the islet cells. Furthermore, the method should be cost-effective and be simple to perform and be suited to routine examinations which are to be performed regularly. Moreover, the method should also be suitable for monitoring the progression of a type 1 diabetes disease and for monitoring the treatment success of such a disease.

At least one embodiment of the invention is achieved by an in vitro assay for early diagnosis of insulin-dependent diabetes mellitus type 1, comprising the following steps: i) determining susceptibility markers for diabetes mellitus type 1 in a biological sample from a symptom-free individual; ii) determining inflammatory markers in the same or in a further biological sample from the individual; iii) determining the presence or the risk of developing insulin-dependent diabetes mellitus type 1 on the basis of the values obtained in i) and ii); and, possibly, iv) performing step ii) after a defined time interval or v) performing an imaging method for the pancreas. At least one embodiment of the invention furthermore relates to the use of this assay for monitoring the progression of a diabetes mellitus type 1 disease and for monitoring the treatment of a diabetes mellitus type 1 disease.

Surprisingly, it was found that a combination of determining susceptibility markers for diabetes and determining inflammatory markers allows early determination of the risk of diabetes mellitus type 1 or early diagnosis of a diabetes mellitus type 1 disease or diagnosis of the onset of a diabetes mellitus type 1 disease. In particular, the assay (assay method) based on this combination allows a risk to be determined for individuals who do not yet have clinically manifest symptoms of a diabetes mellitus type 1 disease. Furthermore, the assay method according to at least one embodiment of the invention allows early diagnosis of a diabetes mellitus type 1 disease at economically justifiable costs with a sufficient level of diagnostic reliability in order to be able to make a therapy decision. The assay method according to at least one embodiment of the invention is therefore very well suited to routine screening for diabetes within the scope of precautionary examinations, particularly for newborn infants. Naturally, such an assay can also be used to determine the presence of a manifest diabetes mellitus type disease. Children with corresponding risk factors are a preferred group amongst the group of individuals at risk of insulin-dependent diabetes mellitus type 1.

The occurrence of the advantages according to at least one embodiment of the invention depends on whether the number of patients who are examined using imaging methods and suffer from side effects (calculated as the number of patients examined by imaging methods multiplied by the incidence of severe side effects) is significantly smaller than the number of those patients who actually benefit from the early detection of diabetes, that is to say the number of, as a result of the examination, correctly positively tested individuals. This value can easily be determined by a person skilled in the art on the basis of the population-typical percentage diabetes risk and the in vitro tests or their limit values used within the scope of embodiments of the present invention.

### BRIEF DESCRIPTION OF THE DRAWINGS

Further advantages, features and properties of the present invention are explained below in more detail with the aid of example embodiments and with reference to the accompanying drawings, in which:

FIG. 1 explains a flow chart for performing the assay according to an embodiment of the invention

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

According to an embodiment of the invention, susceptibility markers for diabetes are first of all determined in a biological sample of a symptom-free individual or on the basis of, for example, an appropriate medical history. Risk factors for a type 1 diabetes disease are, for example, being overweight, a family medical history with first and second degree relatives having diabetes, belonging to a certain ethnic group, high blood pressure or certain hereditary diseases such as different neuropathies or myopathies or forms of hyperinsulism. The chronic use of various medicaments, such as, for example, glucocorticoids, chemo-therapeutics or some antipsychotics, is also accompanied by an increased risk of diabetes.

Examples of susceptibility markers are certain HLA genotypes, in particular the types DR3 and DR4. An example of a suitable marker are the alleles of the HLA-DQB1 gene, the genotypes DQB1*0201 and DR4-DQB1*0302 being considered to be risk factors. Patients with this genetic marker are considered to be high risk patients with a risk of developing diabetes of approximately 5% (over their lifetime or within the first twenty years); however, they constitute less than 2% of the population. This test can therefore significantly reduce the number of people that have to be examined. About 2000 of 100 000 children and adolescents tested who should be subject to further examination were identified by this test. Such a genetic test using methods from molecular diagnostics can be offered at a cost of a few euros, for example on a microarray (biochip). Alternatively, it is possible for different markers, e.g. HLA DR3/DR4, to be used or for a number of markers to be combined together.

The susceptibility markers are determined in a known fashion in a biological sample of the individual. The biological sample can be any biological sample taken from the individual in which the mentioned susceptibility markers are usually detected. The biological sample is preferably blood or a blood fraction, in particular blood serum. It is preferably blood from veins or capillaries. The susceptibility markers are detected in a conventional fashion by tests known to a person skilled in the art, according to the specifications of the producer.

In the second step, there is a regular (e.g. every six months) determination of a marker for the acute inflammation of the islet cells, for example anti-GAD. The limit value for anti-GAD depends on the utilized test and is prescribed by the producer of the test; e.g. the producer Medipan specifies 5 IU/ml. Patients who are recognized as positive in this test for the first time (i.e. having an antibody concentration greater than 5 IU/ml after one or more preceding examinations showing an antibody concentration of <5 IU/ml) have a high risk of developing manifest diabetes over the following few months. However, such tests also have a false positive rate of approximately 2-3%, that is to say in the abovementioned risk group of 2000 examinees, 40-60 healthy individuals will test positive (false positives), while approximately 5-10 (0.25%-0.5%) will actually fall ill. Alternatively, it is possible for different markers to be used, also in combination; for example, antibodies against insulin, islet cell antibodies or antibodies against tyrosine phosphatase. However, since these markers are not completely independent of one another, this can only achieve limited improvements in the false positive rate because examinees who have a false positive test result in respect of one marker will with a high probability also have a false positive test result in the case of another marker.

It is for this reason that the result of this combination of tests cannot be used as an indicator for therapy, as is proposed in, for example, the publication Diabetologica (2001) 44:290-297, since this would lead to a large number of examinees, who would not develop diabetes even without therapy, being subject to therapy. This holds true particularly in the case where immunosuppressive therapies are used which are able to stop an inflammation of the islet cells, but are related to potentially damaging side effects in the patient.

Further examples of inflammatory markers include C-reactive protein, interleukins, cytokines, tumor necrosis factors and various islet cell antibodies such as cytoplasmic islet cell antibodies (ICA), insulin autoantibodies (IAA), glutamate decarboxylase (GAD) antibodies, tyrosine phosphatase (IA-2) antibodies. The inflammatory markers are determined in a known fashion with conventional tests according to the specifications of the producer. A diagnostic test for determining the islet cell autoantibodies is described, for example, in U.S. Pat. No. 5,786,221, the entire contents of which are hereby incorporated herein by reference.

The test result of the abovementioned combined test methods now allows the targeted indication for an imaging examination. Said examination is performed, for example, by using contrast-agent-supported MRI as is described, for example, in Denis et al. (see above). Alternatively, nuclear medicine methods such as PET or SPECT with inflammation-specific radiotracers can also be used, for example SPECT with technetium-marked cells such as granulocytes. However, it is preferable to perform an MRI since this does not burden the patient with ionizing radiation. Here, there is an indication for an MRI if the probability of suffering a severe side effect as a result of the examination is significantly less than the probability of early detection of diabetes.

In the mentioned example, from the originally 100 000 examinees, there would still be approximately 50 false positives examined by imaging (in vitro test positive, but imaging negative) and approximately 10 correct positives, that is to say altogether 60 people. The mathematical probability of side effects is approximately 1 in 1000 and so, mathematically, 0.06 (60/1000) persons would suffer such a side effect, while 10 persons would benefit from the method; thus, the described method has a significant advantage. Even if mild side effects of imaging are taken into account (probability approximately 5%), there still are 10 persons that benefit compared to 3 (60*5%) who suffer a side effect.

By way of example, if other combined in vitro tests would classify 10 000 people as being suitable for an imaging examination, 10 cases of severe side effects and 500 cases of mild side effects would occur, and therefore more examinees would suffer side effects than benefit from the method, and therefore the method could not be performed as described above and there could be no indication for the imaging examination as described.

On the basis of the above specifications, a person skilled in the art can set the patient-specific indication for performing an MRI on the basis of simple considerations geared to said person's specialist knowledge. Alternatively, there can also be an indication for the renewed determination of the inflammatory markers after a defined time interval. The time interval depends on the type of patient and the results of the determination of the inflammatory markers and generally consists of half a year. However, it is also possible for shorter or longer time intervals to be selected, for example three months or a year. According to the invention, this affords the possibility of optimizing the determination of patients with an early form of diabetes; on the other hand, unnecessary examinations are avoided, as a result of which fewer patients are subject to an examination risk and, moreover, examination costs are saved.

Both the susceptibility markers and the inflammatory markers can be determined individually or in combination. The susceptibility markers to be determined are preferably alleles of the HLA-DQB1 gene. The inflammatory markers are preferably anti-GAD. It is preferable for a combination of these markers to be used.

At least one embodiment of the invention also relates to a diagnostic kit which comprises both reagents for determining the susceptibility markers and reagents for determining the inflammatory markers, together with instructions for the evaluation of the determinations. The invention also relates to the use of this kit for early diagnosis of insulin-dependent diabetes mellitus type 1.

The assay method or method according to an embodiment of the invention allows a reliable and timely diagnosis of diabetes mellitus type 1 in the initial stages thereof. As a result of the method according to the invention, an appropriate therapy can be applied in a timely fashion and thus there can be a significant delay in or even a prevention of the development of the complete picture of diabetes type 1. The method according to an embodiment of the invention can also be used for monitoring the progression of the disease. To the extent that an early form of diabetes mellitus type 1 has already been detected as being present, an appropriate therapy can be initiated. The effectiveness of this therapy, that is to say the prevention of the spread of the inflammation of the pancreas, can be monitored using the assay according to the invention, as a result of which a disease-related adaptation of the therapy can be effected at any time. The assay can also determine the severity of a disease which is already present.

The assay according to an embodiment of the invention is also suitable as a screening test for determining clinically inconspicuous persons with a latent risk of type 1 diabetes. In the case of children, such a screening is recommended every 3 to 5 years; in the case of adults approximately every 10 to 20 years. A first examination for early detection of childhood diabetes is expedient when the infant is one or two years old.

The assay according to an embodiment of the invention is preferably performed on a biological sample from a human, but it is also suitable for the examination of arbitrary animals or animal species.

The in vitro assay method according to an embodiment of the invention can also be performed in vivo in combination with, or accompanying, a suitable diabetes therapy. The treatment of diabetes is well known to a person skilled in the art.

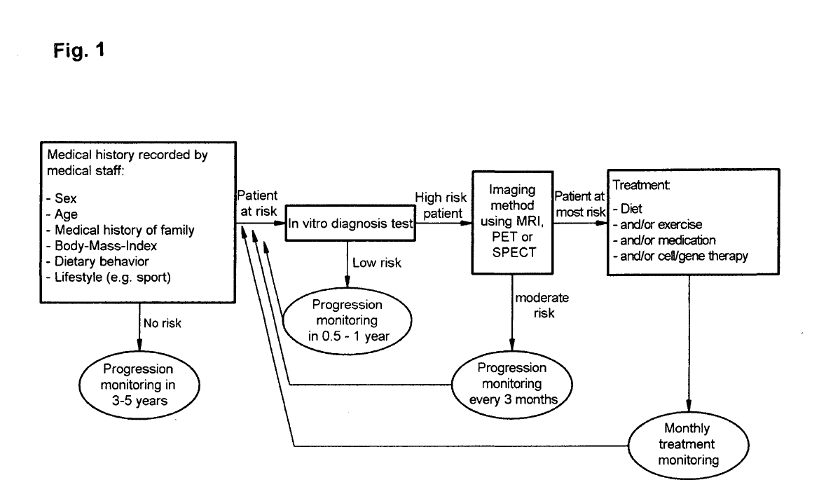

The attached FIG. 1 explains a flow chart for performing the assay according to an embodiment of the invention and the integration thereof into a comprehensive diagnosis and therapy concept for type 1 diabetes.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An in vitro assay for determining a presence or a risk of developing insulin-dependent diabetes mellitus type 1, comprising:
i) determining at least one susceptibility marker for diabetes mellitus type 1 in a biological sample from a symptom-free individual;
ii) determining at least one inflammatory marker in the biological sample or in a further biological sample from the individual;
iii) determining a presence or a risk of developing insulin-dependent diabetes mellitus type 1 based upon values obtained from the determinations in i) and ii).

2. The assay as claimed in claim 1, wherein the at least one susceptibility marker is selected from the HLA genotypes DR3 or DR4.

3. The assay as claimed in claim 1, wherein the at least one inflammatory marker is selected from glutamate decarboxylase antibodies, islet cell antibodies, insulin antibodies or tyrosine phosphatase antibodies.

4. The assay as claimed in claim 1, wherein the biological sample is blood or a blood fraction.

5. The assay as claimed in claim 1, wherein the assay includes performing an imaging method for a pancreas and wherein the imaging method is an inflammation-sensitive MRI scan.

6. A method, comprising:
using the assay, as claimed in claim 1, for monitoring a progression of a diabetes mellitus type 1 disease.

7. A method, comprising:
using the assay, as claimed in claim 1, for monitoring a treatment of a diabetes mellitus type 1 disease.

8. The assay as claimed in claim 2, wherein the at least one inflammatory marker is selected from glutamate decarboxylase antibodies, islet cell antibodies, insulin antibodies or tyrosine phosphatase antibodies.

9. The assay as claimed in claim 2, wherein the biological sample is blood or a blood fraction.

10. The assay as claimed in claim 3, wherein the biological sample is blood or a blood fraction.

11. The assay as claimed in claim 8, wherein the biological sample is blood or a blood fraction.

12. The assay as claimed in claim 1, further comprising:
performing step ii) again after a defined time interval.

13. The assay as claimed in claim 1, further comprising:
performing an imaging method for a pancreas.

14. The assay as claimed in claim 12, further comprising:
performing an imaging method for a pancreas.

15. A method for determining an in vitro assay for determining a presence or a risk of developing insulin-dependent diabetes mellitus type 1, comprising:
i) determining at least one susceptibility marker for diabetes mellitus type 1 in a biological sample from a symptom-free individual;
ii) determining at least one inflammatory marker in the biological sample or in a further biological sample from the individual;
iii) determining a presence or a risk of developing insulin-dependent diabetes mellitus type 1 based upon values obtained from the determinations in i) and ii).

16. A computer readable medium including program segments for, when executed on a computer device, causing the computer device to implement the method of claim 15.

17. A computer readable medium including program segments for, when executed on a computer device, causing the computer device to implement the method of claim 1.
